(19) United States
(12) Patent Application Publication
(10) Pub. No.: US 2021/0355502 A1
(43) Pub. Date: Nov. 18, 2021

(54) MATERIALS AND METHODS FOR THE CORRECTION OF RETINITIS PIGMENTOSA

(71) Applicant: BLUEALLELE, LLC, Maple Grove, MN (US)

(72) Inventor: Nicholas BALTES, Maple Grove, MN (US)

(21) Appl. No.: 17/273,724

(22) PCT Filed: Oct. 2, 2019
(86) PCT No.: PCT/US2019/054294

§ 371 Date: Mar. 4, 2021

Related U.S. Application Data
(60) Provisional application No. 62/830,756, filed on Apr. 8, 2019.
(60) Provisional application No. 62/741,368, filed on Oct. 4, 2018.

(51) Int. Cl. C12N 15/86; C12N 9/22; C12N 15/90

(52) U.S. Cl. CPC C12N 15/86; C12N 9/22; C12N 15/907; A61K 47/6929; C12N 2830/008; C12N 2750/14143; C12N 2830/002

(57) ABSTRACT

Methods and compositions for modifying the coding sequence of endogenous genes using rare-cutting endonucleases. The methods and compositions described herein can be used to modify the endogenous USH2A gene.

### REFERENCE TO RELATED APPLICATIONS

This application claims priority to previously filed and co-pending applications U.S. Ser. No. 62/741,368, filed Oct. 4, 2018 and U.S. Ser. No. 62/830,756, filed Apr. 8, 2019, the contents of which are incorporated herein by reference in their entirety.

### SEQUENCE LISTING

The instant application contains a Sequence Listing which has been submitted in ASCI format via EFS-Web and is hereby incorporated by reference in its entirety. Said ASCII copy, created on Oct. 1, 2019, is named SEQUENCE_LISTING_BA2018-3WO and is 1,286,144 bytes in size.

### TECHNICAL FIELD

The present document is in the field of gene therapy and genome editing.

More specifically, this document relates to the targeted modification of endogenous genes, including the usherin gene, USH2A.

### BACKGROUND

Monogenic disorders are caused by one or more mutations in a single gene, examples of which include sickle cell disease (hemoglobin-beta gene), cystic fibrosis (cystic fibrosis transmembrane conductance regulator gene), and Tay-Sachs disease (beta-hexosaminidase A gene). Monogenic disorders have been an interest for gene therapy, as replacement of the defective gene with a functional copy could provide therapeutic benefits. However, one bottleneck for generating effective therapies includes the size of the functional copy of the gene. Many delivery methods, including those that use viruses, have size limitations which hinder the delivery of large transgenes. Further, many genes have alternative splicing patterns resulting in a single gene coding for multiple proteins. Methods to correct partial regions of a defective gene may provide an alternative means to treat monogenic disorders.

Usher syndrome is an autosomal recessive genetic disorder which causes both retinitis pigmentosa and sensorineural hearing loss, with a prevalence estimated between 3.3 and 6.4 per 100,000 people. Usher syndrome can be divided into three clinical subtypes, depending on severity and onset of symptoms: Usher syndrome type I, Usher type II, and Usher type III. The most common cause of Usher syndrome is caused by mutations in the USH2A gene (75-90% of patients with USH2 have pathogenic mutations in the USH2A gene).

The structure of the USH2A gene, along with the location of pathogenic mutations, have created challenges for generating therapies. The USH2A gene is a relatively large gene, spanning 790 kb and comprising 72 exons. The coding sequence is approximately 15,609 bp, making it too large for current delivery vehicles (e.g., adeno-associated viral vectors). Further, the gene encodes multiple isoforms (Wijk et al., Am. J. Hum. Genet. 74:738-744, 2004), including usherin isoform A (encoded by exons 1-21) and usherin isoform B (encoded by exons 1-72). More than 70 different USH2A alleles harboring pathogenic mutations have been identified (Baux et al., Hum Matat 29:76-87, 2008). Within this study, most of the mutations were present in only one or a few cases, with the exception of the mutation c.2299delG (Glu767fs).

As there are no current treatments available for patients with USH2, development of methods and materials for correcting defective USH2A genes could provide therapeutic options for those with USH2 retinitis pigmentosa.

### SUMMARY

Several challenges exist with developing effective, safe and robust therapies for Usher syndrome type II. Many of these challenges exist due to the complexity of the USH2A gene. USH2A is a relatively large gene, spanning 790 kb and comprising 72 exons. The coding sequence is approximately 15,609 bp, making it too large for most delivery vehicles (e.g., adeno-associated virus vectors for gene augmentation). The gene encodes multiple isoforms, including usherin isoform A (encoded by exons 1-21) and usherin isoform B (encoded by exons 1-72), and mutations causing USH2 are distributed across the coding sequence of both isoforms. This document provides novel approaches for modifying the USH2A gene.

This disclosure herein describes novel approaches for creating effective therapies for retinitis pigmentosa while addressing the challenges associated with the USH2A gene. Further, the methods are compatible with current delivery vehicles (e.g., adeno-associated viruses) and enable correction of mutations throughout the USH2A gene while accounting for the production of both isoforms (isoform a and isoform b) and expression levels of each. This disclosure herein is based at least in part on the discovery that a portion of the coding sequence of the USH2A gene can be substituted with a coding sequence on a transgene. The methods described herein provide a means for introducing sequence changes spanning multiple exons while maintaining the production of both isoforms. For example, the methods teach how to modify the coding sequence spanning from exon 1 up to exon 21 of the endogenous USH2A gene, or any of the exons 1 up to and including exon 21, or a combination thereof. Further, the methods teach how to modify the USH2A coding sequence spanning from exon 22 through exon 72 or any of the exons of 72 down to and including exon 22, or a combination thereof. The methods described herein can be used to 1) correct or introduce genetic modifications in the endogenous USH2A gene, 2) maintain isoform production while correcting mutations found in patients with retinitis pigmentosa, or 3) maintain appropriate expression levels of each isoform while correcting mutations found in patients with retinitis pigmentosa, or any combination thereof. The modifications can be used for applied research (e.g., gene therapy) or basic research (e.g., creation of animal models, or understanding gene function).

In one embodiment, this document features a method for integrating a transgene into the USH2A gene. The method can include transfecting a cell with a rare-cutting endonuclease or transposase which is targeted to the USH2A gene, along with transfecting a transgene. The transgene can integrate into the USH2A gene following cleavage by the rare-cutting endonuclease or transposition by the transposase. The transgene can comprise sequence that is homologous to one or more exons within the USH2A gene, or alternatively, the transgene can encode sequence that is homologous to part of an USH2A protein. The cell being transfected can include an induced pluripotent stem cell (iPSC), an ear cell, or a cell within the retina. In a preferred embodiment, the transgene comprises a partial USH2A coding sequence which encodes a partial USH2A protein. Within this embodiment, the partial USH2A protein is produced by the coding sequence of exons 2-13 or exons 2-21, or any combination of exons between exons 2-13 and exons 2-21 (e.g., exons 2-14, exons 2-15, exons 2-16, exons 2-17, exons 2-18, exons 2-19, and exons 2-20). The cell can be transfected with a transgene comprising exons 2-13 (i.e., exons 2, 3, 4, 5, 6, 7, 8, 9, 10, 11, 12, and 13) of a functional human USH2A gene. Alternatively, the cell can be transfected with a transgene encoding the sequence shown in SEQ ID NO: 13. The transgene can further comprise a promoter which drives expression of the exons. Alternative to a promoter, the transgene can comprise an IRES sequence or a 2A sequence. The transgene can be integrated in an endogenous USH2A gene at the end of exon 13 or within intron 13. In another embodiment, the cell can be transfected with a transgene comprising exons 62-72 (i.e., exons 62, 63, 64, 65, 66, 67, 68, 69, 70, 71, and 72) of the USH2A gene. Alternatively, the cell can be transfected with a transgene encoding the sequence shown in SEQ ID NO: 14. In both cases, the sequence can be followed by a terminator. The transgene can be integrated in an endogenous USH2A gene at the beginning of exon 62 or within intron 61. The rare-cutting endonucleases, which facilitate the integration of the transgene, can include a zinc-finger nuclease, a transcription activator-like effector nuclease, or a CRISPR/Cas endonuclease. The transgene can be delivered to cells using viral vectors, including adenoviral (Ad) vectors, lentiviral vectors, or an adeno-associated viral (AAV) vectors. The transposase which facilitates integration of the transgene can include CRISPR-associated transposase systems. These systems can include Cas12k or Cas6.

In embodiments, the transgene can comprise a partial coding sequence encoding the peptide produced by USH2A exon 2, and the insertion location can be within exon 2, or within intron 3. The transgene can comprise a partial coding sequence encoding the peptide produced by USH2A exons 2 through 3, and the insertion location can be within exon 3, or within intron 4. The transgene can comprise a partial coding sequence encoding the peptide produced by USH2A exons 2 through 4, and the insertion location can be within exon 4, or within intron 5. The transgene can comprise a partial coding sequence encoding the peptide produced by USH2A exons 2 through 5, and the insertion location can be within exon 5, or within intron 6. The transgene can comprise a partial coding sequence encoding the peptide produced by USH2A exons 2 through 6, and the insertion location can be within exon 6, or within intron 7. The transgene can comprise a partial coding sequence encoding the peptide produced by USH2A exons 2 through 7, and the insertion location can be within exon 7, or within intron 8. The transgene can comprise a partial coding sequence encoding the peptide produced by USH2A exons 2 through 8, and the insertion location can be within exon 8, or within intron 9. The transgene can comprise a partial coding sequence encoding the peptide produced by USH2A exons 2 through 9, and the insertion location can be within exon 9, or within intron 10. The transgene can comprise a partial coding sequence encoding the peptide produced by USH2A exons 2 through 10, and the insertion location can be within exon 10, or within intron 11. The transgene can comprise a partial coding sequence encoding the peptide produced by USH2A exons 2 through 11, and the insertion location can be within exon 11, or within intron 12. The transgene can comprise a partial coding sequence encoding the peptide produced by USH2A exons 2 through 12, and the insertion location can be within exon 12, or within intron 13. The transgene can comprise a partial coding sequence encoding the peptide produced by USH2A exons 2 through 13, and the insertion location can be within exon 13, or within intron 14. The transgene can comprise a partial coding sequence encoding the peptide produced by USH2A exons 2 through 14, and the insertion location can be within exon 14, or within intron 15. The transgene can comprise a partial coding sequence encoding the peptide produced by USH2A exons 2 through 15, and the insertion location can be within exon 15, or within intron 16. The transgene can comprise a partial coding sequence encoding the peptide produced by USH2A exons 2 through 16, and the insertion location can be within exon 16, or within intron 17. The transgene can comprise a partial coding sequence encoding the peptide produced by USH2A exons 2 through 17, and the insertion location can be within exon 17, or within intron 18. The transgene can comprise a partial coding sequence encoding the peptide produced by USH2A exons 2 through 18, and the insertion location can be within exon 18, or within intron 19. The transgene can comprise a partial coding sequence encoding the peptide produced by USH2A exons 2 through 19, and the insertion location can be within exon 19, or within intron 20. The transgene can comprise a partial coding sequence encoding the peptide produced by USH2A exons 2 through 20, and the insertion location can be within exon 20, or within intron 21. The transgene can comprise a partial coding sequence encoding the peptide produced by USH2A exons 2 through 21, and the insertion location can be within exon 21, or within intron 22. The transgene can comprise a partial coding sequence encoding the peptide produced by USH2A exons 22 through 72, and the insertion location can be within intron 21, or immediately preceding intron 21 (i.e., at the junction between intron 21 and exon 22). The transgene can comprise a partial coding sequence encoding the peptide produced by USH2A exons 23 through 72, and the insertion location can be within intron 22, or immediately preceding intron 22. The transgene can comprise a partial coding sequence encoding the peptide produced by USH2A exons 24 through 72, and the insertion location can be within intron 23, or immediately preceding intron 23. The transgene can comprise a partial coding sequence encoding the peptide produced by USH2A exons 25 through 72, and the insertion location can be within intron 24, or immediately preceding intron 24. The transgene can comprise a partial coding sequence encoding the peptide produced by USH2A exons 26 through 72, and the insertion location can be within intron 25, or immediately preceding intron 25. The transgene can comprise a partial coding sequence encoding the peptide produced by USH2A exons 27 through 72, and the insertion location can be within intron 26, or immediately preceding intron 26. The transgene can comprise a partial coding sequence encoding the peptide produced by USH2A exons 28 through 72, and the insertion location can be within intron 27, or immediately preceding intron 27. The transgene can comprise a partial coding sequence encoding the peptide produced by USH2A exons 29 through 72, and the insertion location can be within intron 28, or immediately preceding intron 28. The transgene can comprise a partial coding sequence encoding the peptide produced by USH2A exons 30 through 72, and the insertion location can be within intron 29, or immediately preceding intron 29. The transgene can comprise a partial coding sequence encoding the peptide produced by USH2A exons 31 through 72, and the insertion location can be within intron 30, or immediately preceding intron 30. The transgene can comprise a partial coding sequence encoding the peptide produced by USH2A exons 32 through 72, and the insertion location can be within intron 31, or immediately preceding intron 31. The transgene can comprise a partial coding sequence encoding the peptide produced by USH2A exons 33 through 72, and the insertion location can be within intron 32, or immediately preceding intron 32. The transgene can comprise a partial coding sequence encoding the peptide produced by USH2A exons 34 through 72, and the insertion location can be within intron 33, or immediately preceding intron 33. The transgene can comprise a partial coding sequence encoding the peptide produced by USH2A exons 35 through 72, and the insertion location can be within intron 34, or immediately preceding intron 34. The transgene can comprise a partial coding sequence encoding the peptide produced by USH2A exons 36 through 72, and the insertion location can be within intron 35, or immediately preceding intron 35. The transgene can comprise a partial coding sequence encoding the peptide produced by USH2A exons 37 through 72, and the insertion location can be within intron 36, or immediately preceding intron 36. The transgene can comprise a partial coding sequence encoding the peptide produced by USH2A exons 38 through 72, and the insertion location can be within intron 37, or immediately preceding intron 37. The transgene can comprise a partial coding sequence encoding the peptide produced by USH2A exons 39 through 72, and the insertion location can be within intron 38, or immediately preceding intron 38. The transgene can comprise a partial coding sequence encoding the peptide produced by USH2A exons 40 through 72, and the insertion location can be within intron 39, or immediately preceding intron 39. The transgene can comprise a partial coding sequence encoding the peptide produced by USH2A exons 41 through 72, and the insertion location can be within intron 40, or immediately preceding intron 40. The transgene can comprise a partial coding sequence encoding the peptide produced by USH2A exons 42 through 72, and the insertion location can be within intron 41, or immediately preceding intron 41. The transgene can comprise a partial coding sequence encoding the peptide produced by USH2A exons 43 through 72, and the insertion location can be within intron 42, or immediately preceding intron 42. The transgene can comprise a partial coding sequence encoding the peptide produced by USH2A exons 44 through 72, and the insertion location can be within intron 43, or immediately preceding intron 43. The transgene can comprise a partial coding sequence encoding the peptide produced by USH2A exons 45 through 72, and the insertion location can be within intron 44, or immediately preceding intron 44. The transgene can comprise a partial coding sequence encoding the peptide produced by USH2A exons 46 through 72, and the insertion location can be within intron 45, or immediately preceding intron 45. The transgene can comprise a partial coding sequence encoding the peptide produced by USH2A exons 47 through 72, and the insertion location can be within intron 46, or immediately preceding intron 46. The transgene can comprise a partial coding sequence encoding the peptide produced by USH2A exons 48 through 72, and the insertion location can be within intron 47, or immediately preceding intron 47. The transgene can comprise a partial coding sequence encoding the peptide produced by USH2A exons 49 through 72, and the insertion location can be within intron 48, or immediately preceding intron 48. The transgene can comprise a partial coding sequence encoding the peptide produced by USH2A exons 50 through 72, and the insertion location can be within intron 49, or immediately preceding intron 49. The transgene can comprise a partial coding sequence encoding the peptide produced by USH2A exons 51 through 72, and the insertion location can be within intron 50, or immediately preceding intron 50. The transgene can comprise a partial coding sequence encoding the peptide produced by USH2A exons 52 through 72, and the insertion location can be within intron 51, or immediately preceding intron 51. The transgene can comprise a partial coding sequence encoding the peptide produced by USH2A exons 53 through 72, and the insertion location can be within intron 52, or immediately preceding intron 52. The transgene can comprise a partial coding sequence encoding the peptide produced by USH2A exons 54 through 72, and the insertion location can be within intron 53, or immediately preceding intron 53. The transgene can comprise a partial coding sequence encoding the peptide produced by USH2A exons 55 through 72, and the insertion location can be within intron 54, or immediately preceding intron 54. The transgene can comprise a partial coding sequence encoding the peptide produced by USH2A exons 56 through 72, and the insertion location can be within intron 55, or immediately preceding intron 55. The transgene can comprise a partial coding sequence encoding the peptide produced by USH2A exons 57 through 72, and the insertion location can be within intron 56, or immediately preceding intron 56. The transgene can comprise a partial coding sequence encoding the peptide produced by USH2A exons 58 through 72, and the insertion location can be within intron 57, or immediately preceding intron 57. The transgene can comprise a partial coding sequence encoding the peptide produced by USH2A exons 59 through 72, and the insertion location can be within intron 58, or immediately preceding intron 58. The transgene can comprise a partial coding sequence encoding the peptide produced by USH2A exons 60 through 72, and the insertion location can be within intron 59, or immediately preceding intron 59. The transgene can comprise a partial coding sequence encoding the peptide produced by USH2A exons 61 through 72, and the insertion location can be within intron 60, or immediately preceding intron 60. The transgene can comprise a partial coding sequence encoding the peptide produced by USH2A exons 62 through 72, and the insertion location can be within intron 61, or immediately preceding intron 61. The transgene can comprise a partial coding sequence encoding the peptide produced by USH2A exons 63 through 72, and the insertion location can be within intron 62, or immediately preceding intron 62. The transgene can comprise a partial coding sequence encoding the peptide produced by USH2A exons 64 through 72, and the insertion location can be within intron 63, or immediately preceding intron 63. The transgene can comprise a partial coding sequence encoding the peptide produced by USH2A exons 65 through 72, and the insertion location can be within intron 64, or immediately preceding intron 64. The transgene can comprise a partial coding sequence encoding the peptide produced by USH2A exons 66 through 72, and the insertion location can be within intron 65, or immediately preceding intron 65. The transgene can comprise a partial coding sequence encoding the peptide produced by USH2A exons 67 through 72, and the insertion location can be within intron 66, or immediately preceding intron 66. The transgene can comprise a partial coding sequence encoding the peptide produced by USH2A exons 68 through 72, and the insertion location can be within intron 67, or immediately preceding intron 67. The transgene can comprise a partial coding sequence encoding the peptide produced by USH2A exons 69 through 72, and the insertion location can be within intron 68, or immediately preceding intron 68. The transgene can comprise a partial coding sequence encoding the peptide produced by USH2A exons 70 through 72, and the insertion location can be within intron 69, or immediately preceding intron 69. The transgene can comprise a partial coding sequence encoding the peptide produced by USH2A exons 71 through 72, and the insertion location can be within intron 70, or immediately preceding intron 70. The transgene can comprise a partial coding sequence encoding the peptide produced by USH2A exons 72, and the insertion location can be within intron 71, or immediately preceding intron 71.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DESCRIPTION OF DRAWINGS

FIG. 1 is an illustration of the human USH2A genomic sequence. Shown is the genomic region comprising exons 13 through 21 and target sites for transgene integration.

FIG. 2 is an illustration of an adeno-associated vector comprising a promoter operably linked to a synthetic sequence comprising a partial (p) exon 2 through 13 of the USH2A gene. The partial exon comprises solely the coding sequence (i.e., starting at the start codon).

FIG. 3 is an illustration of the method to integrate a transgene comprising a promoter operably linked to exons 2(p)-13 of the USH2A gene into the endogenous USH2A genomic sequence. Also shown is the transcriptional product that is generated after integration occurs.

FIG. 4 is an illustration of the human USH2A genomic sequence. Shown is the genomic region comprising exons 53 through 72 and target sites for transgene integration.

FIG. 5 is an illustration of an adeno-associated vector comprising a terminator operably linked to a synthetic sequence comprising exons 60 through a partial exon 72 of the USH2A gene.

FIG. 6 is an illustration of the method to integrate a transgene comprising a terminator operably linked to a synthetic sequence comprising exons 60 through a partial exon 72 of the USH2A gene into the endogenous USH2A genomic sequence. Also shown is the transcriptional product that is generated after integration occurs.

FIG. 7 is an illustration of the integration of a transgene comprising the hCMV-intron promoter upstream of exons 2(p)-13 of the USH2A gene. Also shown is the location of primers for analyzing the integration event.

FIG. 8 is an image of gels detecting integration of partial USH2A coding sequences within the USH2A gene.

FIG. 9 is a graph showing the expression levels of modified USH2A genes within a population of cells delivered gene editing reagents normalized to an internal control (GAPDH).

### DETAILED DESCRIPTION

Disclosed herein are methods and compositions for modifying the coding sequence of endogenous genes. In some embodiments, the methods include inserting a transgene into an endogenous gene, wherein the transgene provides a partial coding sequence which substitutes for the endogenous gene's coding sequence.

In one embodiment, this document provides a method of integrating a transgene into the USH2A gene, where the method comprises administering a rare-cutting endonuclease or transposase targeted to a site within the USH2A gene, and administering a transgene, wherein the transgene is integrated within the USH2A gene. The transgene can include a promoter, 2A sequence, or an internal ribosome entry site operably linked to a partial USH2A coding sequence. The transgene can be integrated in a cell comprising the USH2A gene. The method can include the use of a CRISPR-associated transposase to integrate the transgene, including those having Cas12k or Cas6. The Cas12k sequence can be from Scytonema hofmanni or Anabaena cylindrica. The rare-cutting endonuclease can be selected from a CRISPR nuclease, TAL effector nuclease, zinc-finger nuclease, or meganuclease. The endogenous USH2A gene can include an aberrant USH2A gene with one or more mutations that cause Usher syndrome type II. The transgene integrated into the USH2A gene can include a promoter, a partial USH2A coding sequence from a functional USH2A gene, and a splice donor. In one embodiment, the partial coding sequence can encode a peptide produced by exon 2 of a functional USH2A gene. The partial coding sequence can encode a peptide as shown in SEQ ID NO:55. The transgene comprising exon 2 of a functional USH2A gene, or encoding the peptide produced by exon 2 of a functional USH2A gene can be integrated within exon 13, within exon 21, or in a region between exon 13 and exon 21. In one embodiment, the partial coding sequence can comprise USH2A exons 2-13, or it can encode for a peptide produced by exons 2-13 of a functional USH2A gene. The peptide sequence can comprise the amino acid sequence within SEQ ID NO:13. This transgene encoding a peptide produced by exons 2-13 of a functional USH2A gene can be integrated in exon 13 or intron 13 of an aberrant USH2A gene. In another embodiment, the partial coding sequence comprises exons 2-21 from a functional USH2A gene, or encodes for the peptide produced by exons 2-21 of a functional USH2A gene. The peptide sequence can comprise the amino acid sequence within SEQ ID NO:57. Here, the transgene can be integrated in exon 21 or intron 22 of the USH2A gene. The splice donor within the transgene comprising exons 2-21 or encoding a peptide produced by exons 2-21 can be the splice donor from intron 21. In an embodiment, the transgenes can be administered to cells by electroporation. In other embodiments, the promoter within the transgenes designed to modify the 5′ end of the USH2A gene can be a tissue specific promoter, inducible promoter, constitutive promoter, or native USH2A promoter. In embodiments, the transgenes described herein can comprise additional sequences to promote integration. The transgenes can comprise a left and right homology arm, a transposon left end and right end or one or more target sites for one or more rare-cutting endonucleases. The transgenes can be administered to a cell within the retina. In an embodiment, the transgenes can be harbored on adeno-associated virus vectors. In an embodiment, the transgenes can be administered with lipid nanoparticles.

In another embodiment, the transgene for integration into the endogenous USH2A gene can comprise a splice acceptor, a partial USH2A coding sequence from a functional USH2A gene, and a terminator. The partial coding sequence can encode a peptide produced by exon 72 of a functional USH2A gene. The peptide produced by exon 72 can be the amino acid show in SEQ ID NO:56. The transgene comprising the peptide produced by exon 72 can be integrated in the USH2A gene within the junction of intron 21 and exon 22, the junction of intron 71 and exon 72, or in a region between the junction of intron 21 and exon 22, and the junction of intron 71 and exon 72. In an embodiment, the transgene comprises a partial USH2A coding sequence which encodes for a peptide produced by exons 64-72 of a functional USH2A gene. The partial coding sequence can encode the peptide as shown in SEQ ID NO:61. The transgene can be integrated within intron 63 of the USH2A gene, or at the junction between intron 63 and exon 64. In another embodiment, the transgene can comprise a partial USH2A coding sequence encoded by exons 60-72 of a functional USH2A gene. The partial coding sequence can encode the peptide as shown in SEQ ID NO:62. The transgene can be integrated within intron 63 of the USH2A gene. In embodiments, the transgenes described herein can comprise additional sequences to promote integration. The transgenes can comprise a left and right homology arm, a transposon left end and right end or one or more target sites for one or more rare-cutting endonucleases. The transgenes can be administered to a cell within the retina. In an embodiment, the transgenes can be harbored on adeno-associated virus vectors. In an embodiment, the transgenes can be administered with lipid nanoparticles.

In another embodiment, this document provides an isolated nucleic acid comprising a promoter operably linked to a partial coding sequence of a functional USH2A gene, a splice donor sequence, and a left and right homology arm, a transposon left end and right end or one or more rare-cutting endonuclease target sites. In another embodiment, this document provides an isolated nucleic acid comprising a 2A sequence or internal ribosome entry site operably linked to a partial coding sequence of a functional USH2A gene, a splice donor sequence, and a left and right homology arm, a transposon left end and right end or one or more rare-cutting endonuclease target sites. The partial USH2A coding sequence can include exon 2 of a functional USH2A gene, or encode a peptide produced by exon 2 of a functional USH2A gene. The partial coding sequence can encode SEQ ID NO:55. In another embodiment, the partial USH2A coding sequence can include exons 2-13 of a functional USH2A gene, or encode for the peptide produced by exons 2-13 of a functional USH2A gene. The partial coding sequence can encode SEQ ID NO:13. In another embodiment, the partial USH2A coding sequence can include exons 2-20 of a functional USH2A gene, or encode for the peptide produced by exons 2-20 of a functional USH2A gene. In another embodiment, the partial USH2A coding sequence can include exons 2-21 of a functional USH2A gene, or encode for the peptide produced by exons 2-21 of a functional USH2A gene. The partial coding sequence can encode SEQ ID NO:57. In an embodiment, the isolated nucleic acid sequence can contain a tissue specific promoter, inducible promoter, a native USH2A promoter, or a constitutive promoter. Specifically, the promoter can be sequence from the native USH2A promoter region. In an embodiment, a splice acceptor sequence can be operably linked to the 2A sequence.

In another embodiment, this document provides an isolated nucleic acid comprising a splice acceptor sequence operably linked to a partial coding sequence of a functional USH2A gene, a terminator, and a left and right homology arm, a transposon left end and right end, or one or more rare-cutting endonuclease target sites. The partial USH2A coding sequence can include exon 72 of a functional USH2A gene, or encode a peptide produced by exon 72 of a functional USH2A gene. The partial coding sequence can encode SEQ ID NO:56. The partial USH2A coding sequence can include exons 64-72 of a functional USH2A gene, or encode for a peptide produced by exons 64-72 of a functional USH2A gene. The partial coding sequence can encode SEQ ID NO:61. The partial USH2A coding sequence can include exons 63-72 of a functional USH2A gene, or encode for a peptide produced by exons 63-72 of a functional USH2A gene. The partial coding sequence can encode SEQ ID NO:59. The partial USH2A coding sequence can include exons 22-72 of a functional USH2A gene, or encode for a peptide produced by exons 22-72 of a functional USH2A gene. The partial coding sequence can encode SEQ ID NO:58.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example. Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999: and METHODS IN MOLECULAR BIOLOGY, Vol. 119. “Chromatin Protocols” (P. B. Becker, ed.) Humana Press, Totowa, 1999.

As used herein, the terms “nucleic acid” and “polynucleotide,” can be used interchangeably. Nucleic acid and polynucleotide can refer to a deoxyribonucleotide or ribonucleotide polymer, in linear or circular conformation, and in either single- or double-stranded form. These terms are not to be construed as limiting with respect to the length of a polymer. The terms can encompass known analogues of natural nucleotides, as well as nucleotides that are modified in the base, sugar and/or phosphate moieties.

The terms “polypeptide,” “peptide” and “protein” can be used interchangeably to refer to amino acid residues covalently linked together. The term also applies to proteins in which one or more amino acids are chemical analogues or modified derivatives of corresponding naturally-occurring amino acids.

The terms “operatively linked” or “operably linked” are used interchangeably and refer to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence but need not be directly adjacent to it. For example, an enhancer is a transcriptional regulatory sequence that is operatively linked to a coding sequence, even though they are not contiguous. Further, a 2A sequence is operatively linked to a coding sequence if the 2A sequence facilitates separation of two peptides produced from a single transcript.

As used herein, the term “cleavage” refers to the breakage of the covalent backbone of a nucleic acid molecule. Cleavage can be initiated by a variety of methods including, but not limited to, enzymatic or chemical hydrolysis of a phosphodiester bond. Cleavage can refer to both a single-stranded nick and a double-stranded break. A double-stranded break can occur as a result of two distinct single-stranded nicks. Nucleic acid cleavage can result in the production of either blunt ends or staggered ends. In certain embodiments, rare-cutting endonucleases are used for targeted double-stranded or single-stranded DNA cleavage.

An “exogenous” molecule can refer to a small molecule (e.g., sugars, lipids, amino acids, fatty acids, phenolic compounds, alkaloids), or a macromolecule (e.g., protein, nucleic acid, carbohydrate, lipid, glycoprotein, lipoprotein, polysaccharide), or any modified derivative of the above molecules, or any complex comprising one or more of the above molecules, generated or present outside of a cell, or not normally present in a cell. Exogenous molecules can be introduced into cells. Methods for the introduction of exogenous molecules into cells can include lipid-mediated transfer, electroporation, direct injection, cell fusion, particle bombardment, calcium phosphate co-precipitation, DEAE-dextran-mediated transfer and viral vector-mediated transfer.

An “endogenous” molecule is a small molecule or macromolecule that is present in a particular cell at a particular developmental stage under particular environmental conditions. An endogenous molecule can be a nucleic acid, a chromosome, the genome of a mitochondrion, chloroplast or other organelle, or a naturally-occurring episomal nucleic acid. Additional endogenous molecules can include proteins, for example, transcription factors and enzymes.

As used herein, a “gene,” refers to a DNA region encoding that encodes a gene product, including all DNA regions which regulate the production of the gene product. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, a “wild type gene” refers to a form of the gene that is present at the highest frequency in a particular population.

An “endogenous gene” refers to a DNA region normally present in a particular cell that encodes a gene product as well as all DNA regions which regulate the production of the gene product.

“Gene expression” refers to the conversion of the information, contained in a gene, into a gene product. A gene product can be the direct transcriptional product of a gene. For example, the gene product can be, but not limited to, mRNA, tRNA, rRNA, antisense RNA, ribozyme, structural RNA, or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Encoding” refers to the conversion of the information contained in a nucleic acid, into a product, wherein the product can result from the direct transcriptional product of a nucleic acid sequence. For example, the product can be, but not limited to, mRNA, tRNA, rRNA, antisense RNA, ribozyme, structural RNA, or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

As used herein, the term “recombination” refers to a process of exchange of genetic information between two polynucleotides. The term “homologous recombination (HR)” refers to a specialized form of recombination that can take place, for example, during the repair of double-strand breaks. Homologous recombination requires nucleotide sequence homology present on a “donor” molecule. The donor molecule can be used by the cell as a template for repair of a double-strand break. Information within the donor molecule that differs from the genomic sequence at or near the double-strand break can be stably incorporated into the cell's genomic DNA.

The term “homologous” as used herein refers to a sequence of nucleic acids or amino acids having similarity to a second sequence of nucleic acids or amino acids. In some embodiments, the homologous sequences can have at least 80% sequence identity (e.g., 81%, 85%, 90%, 95%, 96%, 97%, 98%, or 99%, sequence identity) to one another.

The term “integrating” as used herein refers to the process of adding DNA to a target region of DNA. As described herein, integration can be facilitated by several different means, including non-homologous end joining, homologous recombination, or targeted transposition. By way of example, integration of a user-supplied DNA molecule into a target gene can be facilitated by non-homologous end joining. Here, a targeted-double strand break is made within the target gene and a user-supplied DNA molecule is administered. The user-supplied DNA molecule can comprise exposed DNA ends to facilitate capture during repair of the target gene by non-homologous end joining. The exposed ends can be present on the DNA molecule upon administration (i.e., administration of a linear DNA molecule) or created upon administration to the cell (i.e., a rare-cutting endonuclease cleaves the user-supplied DNA molecule within the cell to expose the ends). In a specific example, integration can occur by homology-independent targeted integration (Suzuki et al., Nature 540:144-149, 2016). In another example, integration occurs though homologous recombination. Here, the user-supplied DNA harbors a left and right homology arm. In another example, integration occurs through transposition. Here, the user-supplied DNA harbors a transposon left and right end.

The term “transgene” as used herein refers to a sequence of nucleic acids that can be transferred to an organism or cell. The transgene may comprise a gene or sequence of nucleic acids not normally present in the target organism or cell. Additionally, the transgene may comprise a gene or sequence of nucleic acids that is normally present in the target organism or cell. A transgene can be an exogenous DNA sequence introduced into the cytoplasm or nucleus of a target cell. In one embodiment, the transgenes described herein contain a partial coding sequence, wherein the partial coding sequence encodes a portion of a protein that is functional, compared to that portion of the protein produced in the host.

The term “target gene” as used herein refers to an endogenous gene that is the target for modification. Further, the target gene can be present in two general forms: a “functional” gene or an “aberrant” gene. A functional target gene refers to gene that comprises a sequence of DNA which has the potential, under appropriate conditions, to encode a functional protein. Further, a functional gene refers to a gene that does not comprise a mutation associated or linked with a corresponding genetic disorder. By way of example, a wild type USH2A gene is considered herein as a functional USH2A gene. On the other hand, an aberrant gene refers to a gene that comprises mutations associated with or linked to a corresponding genetic disorder. The aberrant gene can encode an aberrant protein or can express a protein at reduced levels, as compared to a functional gene. The aberrant protein can be an inactive protein, a protein with reduced activity, or a protein with a gain-of-function mutation. By way of example, a functional USH2A gene can encode a functional USH2A protein as shown in SEQ ID NO:48. Additionally, a functional USH2A gene can encode a functional variant of the USH2A protein as shown in SEQ ID NO:48, so long as the variations are not associated with or linked to a corresponding genetic disorder (i.e., Usher syndrome type II). On the other hand, an aberrant USH2A gene can comprise loss-of-function or gain-of-function mutations which lead to phenotype associated with a genetic disorder. Aberrant USH2A genes can include those found in patients with Usher syndrome type II. Specific examples of functional and aberrant USH2A genes are described in McGee et al., J Med Genet 47:499-506, 2010, Baux et al., Hum Mutat 28:781-789, 2007, and Dreyer et al., Hum Mutal 29:451. doi:10.1002humu.9524, 2008, which are incorporated herein by reference.

The term “partial coding sequence” as used herein refers to a sequence of nucleic acids that encodes a partial protein. The partial coding sequence can encode a protein that comprises one or less amino acids as compared to the wild type protein or functional protein. The partial coding sequence can encode a partial protein with homology to the wild type protein or functional protein. The term “partial USH2A coding sequence” as used herein refers to a sequence of nucleic acids that encodes a partial USH2A protein. The partial USH2A protein has one or less amino acids compared to a wild type USH2A protein. The one or less amino acids can be from the N- or C-terminus end of the protein. If the partial USH2A coding sequence is designed to amend the 5′ end of the USH2A gene (i.e., the N-terminus of the USH2A protein), then the partial USH2A coding sequence can encode a minimum of the first 161 amino acids (i.e., the coding region of the second exon) of the USH2A protein. The partial USH2A coding sequence can comprise the first 936 amino acids (i.e., exons 2-13). The partial USH2A coding sequence can comprise a maximum of first 1542 amino acids of the USH2A protein (i.e., the coding region of exon 2 to exon 21). The methionine at position 1 of the partial USH2A protein can be removed from the partial coding sequence when using 2A sequences. The first 161 amino acids can be the amino acids shown in SEQ ID NO:55. The first 936 amino acids can be the amino acids shown in SEQ ID NO:13. The first 1542 amino acids can be the amino acids shown in SEQ ID NO:57. A representative USH2A gene, including intron and exon sequences and boundaries, can be found in NCBI reference sequence, NG_009497.1. A representative USH2A gene sequence can be found in SEQ ID NO:63. The sequence found in SEQ ID NO:63 can be referenced to identify the following exon and intron sequences: exon 1 includes the sequence from 5001 to 5183, exon 2 includes the sequence from 5857 to 6545, exon 3 includes the sequence from 9718 to 9883, exon 4 includes the sequence from 63312 to 63444, exon 5 includes the sequence from 100743 to 100806, exon 6 includes the sequence from 102798 to 103092, exon 7 includes the sequence from 104045 to 104229, exon 8 includes the sequence from 104702 to 104923, exon 9 includes the sequence from 106421 to 106514, exon 10 includes the sequence from 136027 to 136222, exon 11 includes the sequence from 138987 to 139117, exon 12 includes the sequence from 177299 to 177494, exon 13 includes the sequence from 181171 to 181812, exon 14 includes the sequence from 196261 to 196444, exon 15 includes the sequence from 210847 to 211010, exon 16 includes the sequence from 220966 to 221124, exon 17 includes the sequence from 228276 to 228770, exon 18 includes the sequence from 229813 to 230082, exon 19 includes the sequence from 231675 to 231844, exon 20 includes the sequence from 238030 to 238174, exon 21 includes the sequence from 252915 to 253145, exon 22 includes the sequence from 331184 to 331314, exon 23 includes the sequence from 339258 to 339384, exon 24 includes the sequence from 341577 to 341678, exon 25 includes the sequence from 343520 to 343699, exon 26 includes the sequence from 344811 to 344941, exon 27 includes the sequence from 350035 to 350308, exon 28 includes the sequence from 355097 to 355300, exon 29 includes the sequence from 355428 to 355508, exon 30 includes the sequence from 358105 to 358296, exon 31 includes the sequence from 379750 to 379863, exon 32 includes the sequence from 381805 to 381966, exon 33 includes the sequence from 427835 to 427994, exon 34 includes the sequence from 429339 to 429510, exon 35 includes the sequence from 435230 to 435377, exon 36 includes the sequence from 457621 to 457772, exon 37 includes the sequence from 462918 to 463080, exon 38 includes the sequence from 493602 to 493781, exon 39 includes the sequence from 527492 to 527642, exon 40 includes the sequence from 528180 to 528322, exon 41 includes the sequence from 539343 to 539971, exon 42 includes the sequence from 549299 to 549633, exon 43 includes the sequence from 550517 to 550639, exon 44 includes the sequence from 561227 to 561390, exon 45 includes the sequence from 582364 to 582573, exon 46 includes the sequence from 583901 to 584103, exon 47 includes the sequence from 590294 to 590406, exon 48 includes the sequence from 611202 to 611400, exon 49 includes the sequence from 614493 to 614661, exon 50 includes the sequence from 629272 to 629490, exon 51 includes the sequence from 638115 to 638338, exon 52 includes the sequence from 641523 to 641727, exon 53 includes the sequence from 645462 to 645659, exon 54 includes the sequence from 646201 to 646355, exon 55 includes the sequence from 648356 to 648554, exon 56 includes the sequence from 661609 to 661716, exon 57 includes the sequence from 668554 to 668737, exon 58 includes the sequence from 669645 to 669802, exon 59 includes the sequence from 685062 to 685220, exon 60 includes the sequence from 686860 to 687022, exon 61 includes the sequence from 700013 to 700367, exon 62 includes the sequence from 748021 to 748248, exon 63 includes the sequence from 752781 to 754297, exon 64 includes the sequence from 757104 to 757425, exon 65 includes the sequence from 777596 to 777805, exon 66 includes the sequence from 779631 to 779869, exon 67 includes the sequence from 780667 to 780875, exon 68 includes the sequence from 787663 to 787839, exon 69 includes the sequence from 789159 to 789242, exon 70 includes the sequence from 793694 to 793938, exon 71 includes the sequence from 799362 to 799583, exon 72 includes the sequence from 802527 to 805503, intron 1 includes the sequence from 5184 to 5856, intron 2 includes the sequence from 6546 to 9717, intron 3 includes the sequence from 9884 to 63311, intron 4 includes the sequence from 63445 to 100742, intron 5 includes the sequence from 100807 to 102797, intron 6 includes the sequence from 103093 to 104044, intron 7 includes the sequence from 104230 to 104701, intron 8 includes the sequence from 104924 to 106420, intron 9 includes the sequence from 106515 to 136026, intron 10 includes the sequence from 136223 to 138986, intron 11 includes the sequence from 139118 to 177298, intron 12 includes the sequence from 177495 to 181170, intron 13 includes the sequence from 181813 to 196260, intron 14 includes the sequence from 196445 to 210846, intron 15 includes the sequence from 211011 to 220965, intron 16 includes the sequence from 221125 to 228275, intron 17 includes the sequence from 228771 to 229812, intron 18 includes the sequence from 230083 to 231676, intron 19 includes the sequence from 231845 to 238029, intron 20 includes the sequence from 238175 to 252914, intron 21 includes the sequence from 253146 to 331183, intron 22 includes the sequence from 331315 to 339257, intron 23 includes the sequence from 339385 to 341576, intron 24 includes the sequence from 341679 to 343519, intron 25 includes the sequence from 343700 to 344810, intron 26 includes the sequence from 344942 to 350034, intron 27 includes the sequence from 350309 to 355096, intron 28 includes the sequence from 355301 to 355427, intron 29 includes the sequence from 355509 to 358104, intron 30 includes the sequence from 358297 to 379749, intron 31 includes the sequence from 379864 to 381804, intron 32 includes the sequence from 381967 to 427834, intron 33 includes the sequence from 427995 to 429338, intron 34 includes the sequence from 429511 to 435229, intron 35 includes the sequence from 435378 to 457620, intron 36 includes the sequence from 457773 to 462917, intron 37 includes the sequence from 463081 to 493601, intron 38 includes the sequence from 493782 to 527491, intron 39 includes the sequence from 527643 to 528179, intron 40 includes the sequence from 528323 to 539342, intron 41 includes the sequence from 539972 to 549298, intron 42 includes the sequence from 549634 to 550516, intron 43 includes the sequence from 550640 to 561226, intron 44 includes the sequence from 561391 to 582363, intron 45 includes the sequence from 582574 to 583900, intron 46 includes the sequence from 584104 to 590293, intron 47 includes the sequence from 590407 to 611201, intron 48 includes the sequence from 611401 to 614492, intron 49 includes the sequence from 614662 to 629271, intron 50 includes the sequence from 629491 to 638114, intron 51 includes the sequence from 638339 to 641522, intron 52 includes the sequence from 641728 to 645461, intron 53 includes the sequence from 645660 to 646200, intron 54 includes the sequence from 646356 to 648355, intron 55 includes the sequence from 648555 to 661608, intron 56 includes the sequence from 661717 to 668553, intron 57 includes the sequence from 668738 to 669644, intron 58 includes the sequence from 669803 to 685061, intron 59 includes the sequence from 685221 to 686859, intron 60 includes the sequence from 687023 to 700012, intron 61 includes the sequence from 700368 to 748020, intron 62 includes the sequence from 748249 to 752780, intron 63 includes the sequence from 754298 to 757103, intron 64 includes the sequence from 757426 to 777595, intron 65 includes the sequence from 777806 to 779630, intron 66 includes the sequence from 779870 to 780666, intron 67 includes the sequence from 780876 to 787662, intron 68 includes the sequence from 787840 to 789158, intron 69 includes the sequence from 789243 to 793693, intron 70 includes the sequence from 793939 to 799361, and intron 71 includes the sequence from 799584 to 802526.

If the partial USH2A coding sequence is designed to amend the 3′ end of the USH2A gene (i.e., the C-terminus of the USH2A protein), then the partial USH2A coding sequence can encode a minimum of the last 29 amino acids (i.e., the coding region in the last exon) of the USH2A protein, and a maximum of last 2795 amino acids of the USH2A protein (i.e., the coding region of exon 22 to exon 72). The partial USH2A coding sequence can comprise the last 1104 amino acids of the USH2A protein (i.e., exons 64-72). The last 29 amino acids can be the amino acids shown in SEQ ID NO:56. The last 3659 amino acids can be the amino acids shown in SEQ ID NO:58. The last 1104 amino acids can be the amino acids shown in SEQ ID NO:59.

An embodiment provides for the transgene producing a functional fragment of the polypetide. A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

The transgene can also include “functional variants” of the USH2A gene disclosed. Functional variants include, for example, sequences having one or more nucleotide substitutions, deletions or insertions and wherein the variant retains functional polypeptide. Functional variants can be created by any of a number of methods available to one skilled in the art, such as by site-directed mutagenesis, induced mutation, identified as allelic variants, cleaving through use of restriction enzymes, or the like. Examples of functional variants for USH2A include those described in McGee et al., J Med Genet 47:499-506, 2010. These can include, but are not limited to, Lys2080Asn, Ser2196Thr, Val2562Ala, Arg2573His, Asn2930Lys, Thr3115Ala, Asn3199Asp, Gly3618Ser, Arg4192His, Arg4570His, Gly4838Glu, Thr4844Met, Arg4848Gln, Lys5026Glu, and Val5145Ile.

The term “transposase” as used herein refers to one or more proteins that facilitate the integration of a transposon. A transposase can include a CRISPR-associated transposase (Strecker et al., Science 10.1126/science.aax9181, 2019; Klompe et al., Nature, 10.1038/s41586-019-1323-z, 2019). The transposases can be used in combination with a transgene comprising a transposon left end and right end. The CRISPR transposases can include the TypeV-U5, C2C5 CRISPR protein, Cas12k, along with proteins tnsB, tnsC, and tniQ. In some embodiments, the Cas12k can be from Scytonema hofmanni (SEQ ID NO:21) or Anabaena cylindrica (SEQ ID NO:22). Alternatively, the CRISPR transposase can include the Cas6 protein, along with helper proteins including Cas7, Cas8 and TniQ.

The terms “left end” and “right end” as used herein refers to a sequence of nucleic acids present on a transposon, which facilitates integration by a transposase. By way of example, integration of DNA using ShCas12k can be facilitated through a left end (SEQ ID NO:23) and right end sequence (SEQ ID NO:24) flanking a cargo sequence.

As used herein, the term “lipid nanoparticle” refers to a transfer vehicle comprising one or more lipids. The term “lipid nanoparticle” also refers to particles having at least one dimension on the order of nanometers (e.g., 1-1,000 nm) which include one or more lipids. The one or more lipids can be cationic lipids, non-cationic lipids, or PEG-modified lipids. The lipid nanoparticles can be formulated to deliver one or more gene editing reagents to one or more target cells. Examples of suitable lipids include phosphatidylglycerol, phosphatidylcholine, phosphatidylserine, phosphatidylethanolamine, sphingolipids, cerebrosides, and gangliosides. Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a gene editing reagent to a target cell.

The term “region between exon 13 and 21” refers to a location within the USH2A gene. This location includes the sequence spanning from the first nucleotide of intron 13 to the last nucleotide of intron 20. For example, in the representative USH2A gene as shown in SEQ ID NO:63, the “region between exon 13 and 21” corresponds to the nucleotides 181813-252914.

The term “administering” refers to making application of or giving. To administer a rare-cutting endonuclease refers to making an application of, or giving, a rare-cutting endonuclease. A rare-cutting endonuclease can be administered to a cell, which refers to giving (i.e., delivering) the rare-cutting endonuclease to a cell. A rare-cutting endonuclease can be delivered to a cell through different formats, including nucleic acid, either RNA or DNA (also referred to as polynucleotides), which encodes the rare-cutting endonuclease, or purified protein, or a mixture of RNA, DNA or purified protein. Also, the rare-cutting endonuclease can be delivered by viral vectors. Delivery can be achieved through any suitable method, including electroporation, lipofection, biolistics, or sonication.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1: -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt): -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. The percent sequence identity value is rounded to the nearest tenth.

In one embodiment, the methods include modifying an endogenous usherin (USH2A) gene. The modification can be the insertion of a transgene in the endogenous USH2A genomic sequence. The transgene can include a synthetic and partial coding sequence for the USH2A protein. The partial coding sequence can be homologous to coding sequence within a wild type USH2A gene, or a functional variant of the wild type USH2A gene, or a mutant of the wild type USH2A gene. In some embodiments, the transgene encoding the partial USH2A protein is inserted into the 5′ end of an endogenous USH2A genomic sequence but before intron 21 (i.e., within exons or introns 1-21 but before intron 21). The transgene within the 5′ end of the USH2A gene can harbor a promoter and a synthetic and partial USH2A coding sequence that functions to replace the endogenous exons present upstream of the site of integration. Alternative to a promoter, the transgene can harbor a 2A sequence or an internal ribosome entry site. In other embodiments, the transgene encoding the partial USH2A protein is inserted into the 3′ end of an endogenous USH2A genomic sequence (i.e., within exons or introns 22-72). The transgene within the 3′ end of the USH2A gene can harbor a terminator and a synthetic and partial USH2A coding sequence that functions to replace the endogenous exons present downstream of the site of integration. The methods described herein can be used to modify regions of the coding sequence for endogenous genes, including the USH2A gene.

In other embodiments, the methods described in this document provide transgenes for integration within the 5′ end of an endogenous USH2A. The transgenes can comprise a promoter and a synthetic and partial USH2A coding sequence, which function to replace the endogenous exons present upstream of the site of integration. In other embodiments, the transgene can comprise a sequence coding for an internal ribosome entry site (IRES) or a self-cleaving 2A peptide followed by the partial USH2A coding sequence. The 2A peptide sequence can be a T2A peptide, a P2A peptide, an E2A peptide, an F2A peptide, or any sequence that results the separation of two polypeptides from a single open reading frame. The 1RES sequence can be a poliovirus IRES, an HCV IRES, an HIV IRES, a p53 IRES, an XIAP IRES, a Bcl-2 IRES, or any sequence that can attract ribosomes in a cap-independent manner.

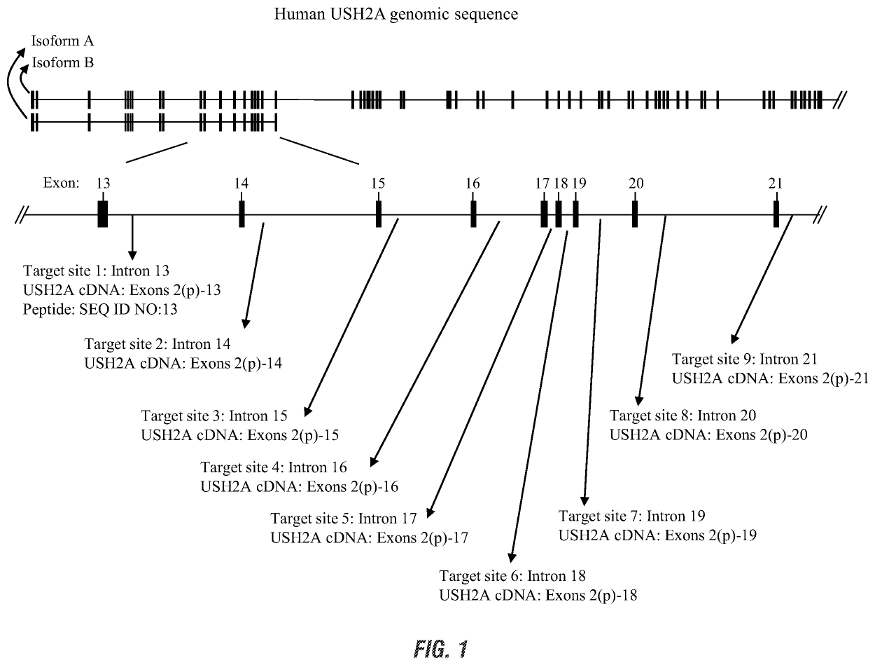

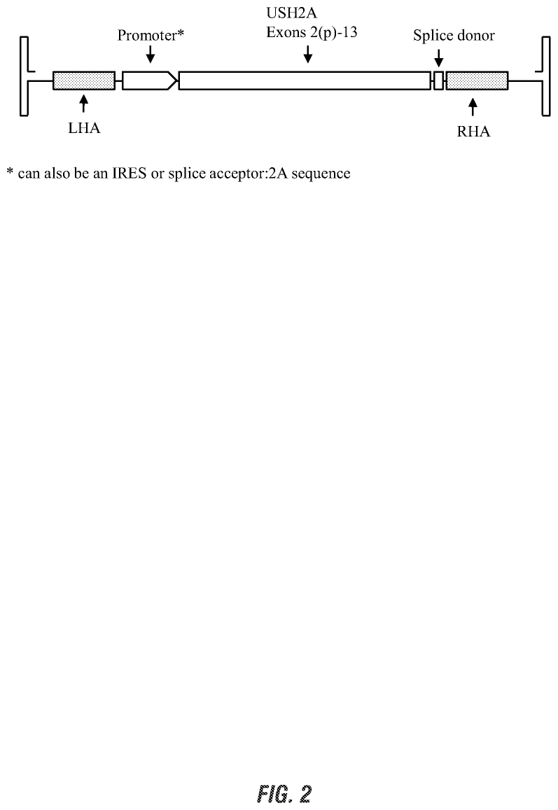

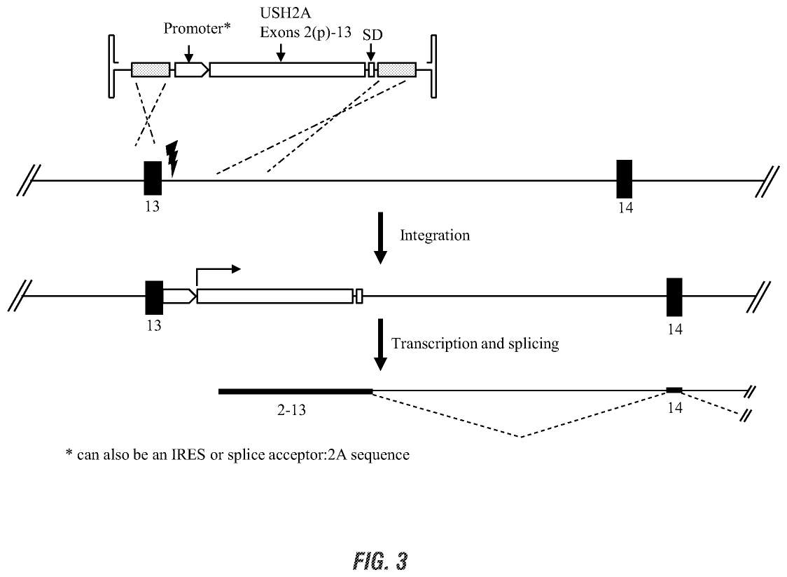

In one embodiment, the methods and compositions described herein can be used to modify the 5′ end of the USH2A coding sequence, thereby resulting in modification of the N-terminus of the USH2A protein. The modification of the 5′ end of the USH2A coding sequence can include the replacement of exon 1 up to exon 21. The modification can include exons 1-21, or 1-20, or 1-19, or 1-18, or 1-17, or 1-16, or 1-15, or 1-14, or 1-13, or 1-12, or 1-11, or 1-10, or 1-9, or 1-8, or 1-7, or 1-6, or 1-5, or 1-4, or 1-3, or 1-2, or 2-21, or 2-20, or 2-19, or 2-18, or 2-17, or 2-16, or 2-15, or 2-14, or 2-13, or 2-12, or 2-11, or 2-10, or 2-9, or 2-8, or 2-7, or 2-6, or 2-5, or 2-4, or 2-3 or 2. In one embodiment, the method to modify the 5′ end of the USH2A coding sequence includes the integration of a transgene into the endogenous USH2A gene. The transgene can harbor a partial synthetic USH2A coding sequence comprising exons 1-21, or 1-20, or 1-19, or 1-18, or 1-17, or 1-16, or 1-15, or 1-14, or 1-13, or 1-12, or 1-11, or 1-10, or 1-9, or 1-8, or 1-7, or 1-6, or 1-5, or 1-4, or 1-3, or 1-2 or 2-21, or 2-20, or 2-19, or 2-18, or 2-17, or 2-16, or 2-15, or 2-14, or 2-13, or 2-12, or 2-11, or 2-10, or 2-9, or 2-8, or 2-7, or 2-6, or 2-5, or 2-4, or 2-3 or 2. The transgene harboring the partial synthetic USH2A coding sequence can be integrated within the endogenous USH2A gene at a site that is within or downstream of the exon which corresponds to the last exon of the partial synthetic coding sequence (FIG. 1). The synthetic USH2A coding sequence can also comprise a promoter, IRES, or 2A sequence operably linked to the synthetic USH2A coding sequence. The synthetic USH2A coding sequence can also comprise a splice donor sequence which facilitates the splicing of the intron between the last exon within the synthetic USH2A coding sequence and the downstream exon within the endogenous USH2A sequence (FIGS. 2 and 3). The transgene can be designed in a donor molecule with arms of homology to a target site. The donor molecule can be incorporated into an AAV vector and particle, and delivered in vivo to target cells. The target cells can comprise a USH2A gene with either low or high gene expression. The target cells can be, for example, induced pluripotent stem cells, ear cells or retinal cells. The AAV comprising the donor molecule can be delivered with or without a second AAV encoding a rare-cutting endonuclease. The second AAV encoding a rare-cutting endonuclease can be used to facilitate integration of the donor molecule with the endogenous USH2A gene.

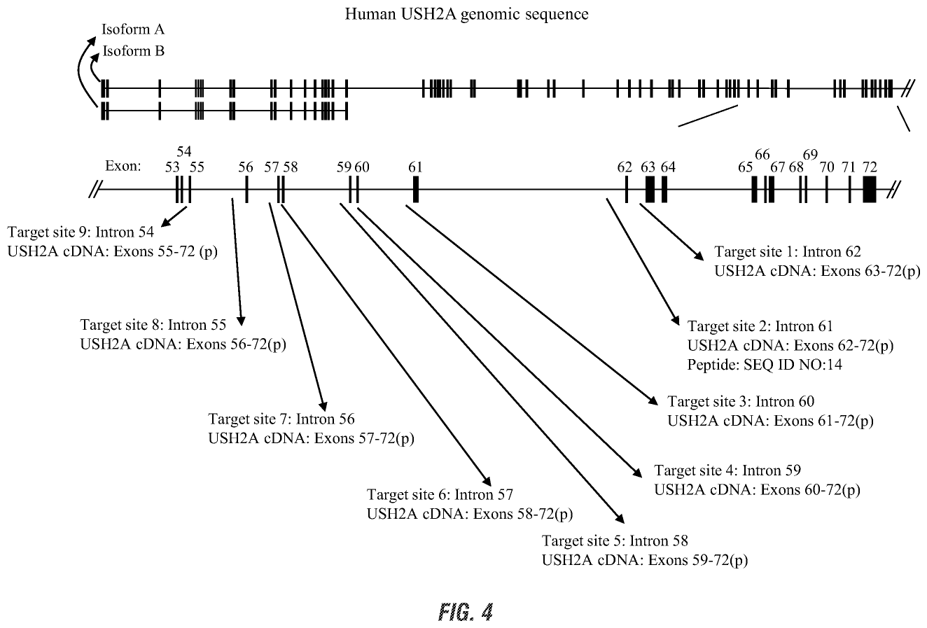

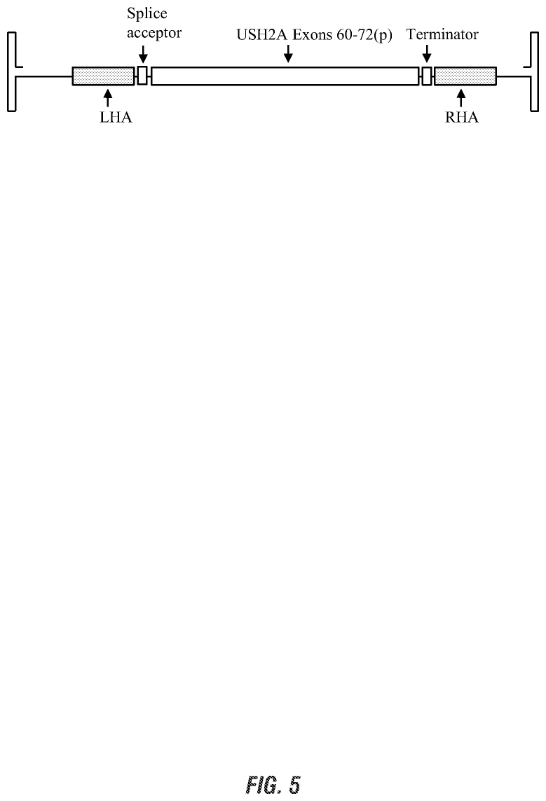

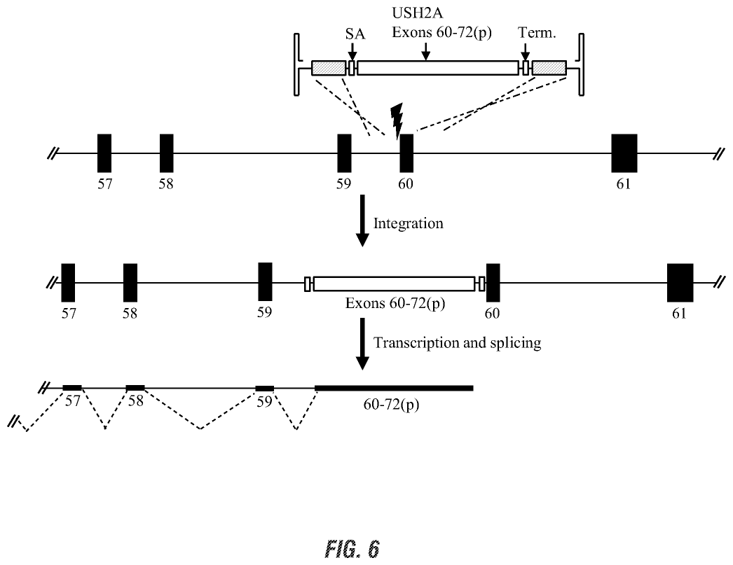

In another embodiment, the methods and compositions described herein can be used to modify the 3′ end of the USH2A coding sequence, thereby resulting in modification of the C-terminus of the USH2A protein. The modification of the 3′ end of the USH2A coding sequence can include the replacement of exon 72 down to exon 22. The modification of the 3′ end of the USH2A coding sequence can include the replacement of exons 22-72, or 23-72, or 24-72, or 25-72, or 26-72, or 27-72, or 28-72, or 29-72, or 30-72, or 31-72, or 32-72, or 33-72, or 34-72, or 35-72, or 36-72, or 37-72, or 38-72, or 39-72, or 40-72, or 41-72, or 42-72, or 43-72, or 44-72, or 45-72, or 46-72, or 47-72, or 48-72, or 49-72, or 50-72, or 51-72, or 52-72, or 53-72, or 54-72, or 55-72, or 56-72, or 57-72, or 58-72, or 59-72, or 60-72, or 61-72, or 62-72, or 63-72, or 64-72, or 65-72, or 66-72, or 67-72, or 68-72, or 69-72, or 70-72, or 71-72 or 72. In one embodiment, the method to modify the 3′ end of the USH2A coding sequence includes the integration of a transgene into the endogenous USH2A gene. The transgene can harbor a partial synthetic USH2A coding sequence comprising exons 22-72, or 23-72, or 24-72, or 25-72, or 26-72, or 27-72, or 28-72, or 29-72, or 30-72, or 31-72, or 32-72, or 33-72, or 34-72, or 35-72, or 36-72, or 37-72, or 38-72, or 39-72, or 40-72, or 41-72, or 42-72, or 43-72, or 44-72, or 45-72, or 46-72, or 47-72, or 48-72, or 49-72, or 50-72, or 51-72, or 52-72, or 53-72, or 54-72, or 55-72, or 56-72, or 57-72, or 58-72, or 59-72, or 60-72, or 61-72, or 62-72, or 63-72, or 64-72, or 65-72, or 66-72, or 67-72, or 68-72, or 69-72, or 70-72, or 71-72 or 72. The partial synthetic USH2A coding sequence can be integrated within the endogenous USH2A gene upstream or within the exon which corresponds to the first exon within the partial synthetic USH2A coding sequence (FIG. 4). The synthetic USH2A coding sequence can comprise a terminator linked to the last exon in the synthetic USH2A coding sequence. The partial synthetic USH2A coding sequence can also comprise a splice acceptor sequence which facilitates the splicing of the intron between the first exon within the synthetic USH2A coding sequence and the upstream exon within the endogenous USH2A sequence (FIGS. 5 and 6). The transgene can be designed in a donor molecule with arms of homology to the target sequence. The donor molecule can be incorporated into an AAV vector and particle, and delivered in vivo to target cells. The target cells can comprise an endogenous USH2A gene with moderate to high expression. The target cells can be, for example, induced pluripotent stem cells, ear cells or retinal cells. The AAV comprising the donor molecule can be delivered with or without a second AAV encoding a rare-cutting endonuclease. The second AAV encoding a rare-cutting endonuclease can be used to facilitate recombination of the donor molecule with the endogenous USH2A gene.

In one embodiment, the methods described herein involve the integration of a promoter, partial USH2A coding sequence, and splice donor sequence into the USH2A gene. The promoter within the transgene can be a constitutive promoter, tissue specific promoter, inducible promoter or the native USH2A promoter. The constitutive promoter can be, but not limited to, a CMV promoter, an EF1a promoter, an SV40 promoter, a PGK1 promoter, a Ubc promoter, a human beta actin promoter, or a CAG promoter. The inducible promoter can be, but not limited to, the tetracycline-dependent regulatable promoters or steroid hormone receptor promoters, including the promoters for the progesterone receptor regulatory system. The inducible promoter can be based upon ecdysone-based inducible systems, progesterone-based inducible systems, estrogen-based inducible systems, CID-(chemical inducers of dimerization) based systems or IPTG-based inducible systems. In one embodiment, the transgene comprising an inducible promoter, partial USH2A coding sequence and splice donor sequence is integrated within the endogenous USH2A gene in cells. To enable expression of the modified USH2A gene, the cells are also administered any necessary nucleic acid or proteins to complete the system (e.g., the chimeric regulator GLVP for progesterone-based inducible systems) and are exposed to the inducer (e.g., RU486). In an embodiment, the native USH2A promoter can be operably linked to the partial USH2A coding sequence. The promoter can include sequence upstream of the USH2A 5′ UTR sequence. The promoter can include the upstream 500 nucleotides before the 5′ UTR sequence. Further, the promoter can include the upstream 1000, 1500, 2000, 2500, 3000, 3500, 4000, 4500, 5000, 6000, 7000, 8000, 9000 or 10000 nucleotides before the 5′ UTR sequence.

In one embodiment, the methods described herein involve the integration of a 2A sequence operably linked to a partial USH2A coding sequence which is operably linked to a splice donor sequence. In one embodiment, the transgene is integrated into an exon. When integrated into an exon, the 2A sequence can be integrated such that the 2A sequence matches the coding frame of the upstream coding sequence of the endogenous gene, resulting in translation through the transgene within the correct frame. If the transgene is to be integrated into an intron, the transgene can further comprise a splice acceptor operably linked to the 2A sequence. To ensure translation through the 2A sequence, 0, 1 or 2 or more nucleotides can be added to the spacer between the splice acceptor sequence and 2A sequence. The use of 2A sequences can be used within transgenes designed to correct the 5′ end of the USH2A gene (exons 1-21), particularly for fixing USH2A genes that are defective due to mutations caused by in frame deletions and amino acid substitutions. The use of 2A sequences can be used to correct mutations caused by a frameshift mutation, with limitations. If there is a premature stop codon caused by a frameshift mutation in an exon (and the stop codon is present in the same exon), then the transgene needs to be integrated within the exon comprising the frameshift mutation, and integrated before or within the premature stop codon. For example, if a transgene is designed to repair the c.2299delG mutation in exon 13, and the transgene comprises a 2A sequence operably linked to a functional USH2A coding sequence, then the transgene should be integrated within the sequence shown in SEQ ID NO:60. In the scenario that the frameshift spans an intron before reaching a stop codon, then the transgene can be integrated i) in the exon comprising the mutation, ii) the intron following the exon with the mutation, or iii) the sequence within the following exon but before or within the premature stop codon.

As described herein, the partial coding sequence can comprise 5′ or 3′ UTR sequences. The 5′ or 3′ UTR sequences can be homologous to the endogenous USH2A 5′ and 3′ UTR sequences. In other embodiments, the 5′ and 3′ UTRs can be from other genes, but operably linked to the partial coding sequence on the transgenes described herein.

As described herein, the donor molecule can be in the form of circular or linear double-stranded or single stranded DNA. The donor molecule can be conjugated or associated with a reagent that facilitates stability or cellular update. The reagent can be lipids, calcium phosphate, cationic polymers, DEAE-dextran, dendrimers, polyethylene glycol (PEG) cell penetrating peptides, gas-encapsulated microbubbles or magnetic beads. The donor molecule can be incorporated into a viral particle. The virus can be retroviral, adenoviral, adeno-associated vectors (AAV), herpes simplex, pox virus, hybrid adenoviral vector, epstein-bar virus, lentivirus, or herpes simplex virus.

In certain embodiments, the AAV vectors as described herein can be derived from any AAV. In certain embodiments, the AAV vector is derived from the defective and nonpathogenic parvovirus adeno-associated type 2 virus. All such vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3, 1998; Kearns et al., Gene Ther. 9:748-55, 1996). Other AAV serotypes, including AAV1, AAV2, AAV3, AAV4, AAV5, AAV6, AAV7, AAV8, AAV9 and AAVrh.10 and any novel AAV serotype can also be used in accordance with the present invention. In some embodiments, chimeric AAV is used where the viral origins of the long terminal repeat (LTR) sequences of the viral nucleic acid are heterologous to the viral origin of the capsid sequences. Non-limiting examples include chimeric virus with LTRs derived from AAV2 and capsids derived from AAV5, AAV6, AAV8 or AAV9 (i.e. AAV2/5, AAV2/6, AAV2/8 and AAV2/9, respectively).

The constructs described herein may also be incorporated into an adenoviral vector system. Adenoviral based vectors are capable of very high transduction efficiency in many cell types and do not require cell division. With such vectors, high titer and high levels of expression can been obtained.

For delivery of gene editing reagents to eye or ear cells, systemic modes of administration can be employed, including oral and parenteral routes. Parenteral routes include, by way of example, intravenous, intrarterial, intramuscular, intradermal, subcutaneous, intranasal, and intraperitoneal routes. Gene editing reagents administered systemically may be modified or formulated to target the components to the eye or inner ear. Alternatively, local modes of administration can be used for delivery of gene editing reagents. These include, but are not limited to, intraocular, intraorbital, subconjuctival, intravitreal, subretinal, transscleral or introcochlear routes. In an embodiment, components described herein are delivered subretinally. e.g., by subretinal injection. Subretinal injections may be made directly into the macular, e.g., submacular injection.

In an embodiment, components described herein are delivered by intravitreal injection. In an embodiment, nanoparticle or viral particles are delivered intravitreally. In an embodiment, components described herein are delivered into the inner ear by intracochlear injection.

The methods and compositions of the invention can also be used in the production of modified organisms. The modified organisms can be small mammals, companion animals, livestock, and primates. Non-limiting examples of rodents may include mice, rats, hamsters, gerbils, and guinea pigs. Non-limiting examples of companion animals may include cats, dogs, rabbits, hedgehogs, and ferrets. Non-limiting examples of livestock may include horses, goats, sheep, swine, llamas, alpacas, and cattle. Non-limiting examples of primates may include capuchin monkeys, chimpanzees, lemurs, macaques, marmosets, tamarins, spider monkeys, squirrel monkeys, and vervet monkeys. The methods and compositions of the invention may also be used in zebrafish.

The methods and compositions described herein can be used to facilitate transgene integration in an endogenous USH2A gene. Integration can occur through homologous recombination or non-homologous end joining. To facilitate homologous recombination between the USH2A gene and a donor molecule, the donor molecule can contain sequence that is homologous to the USH2A gene (e.g., exhibiting between about 80 to 100% sequence identity). To further facilitate homologous recombination, a double-strand break or single-strand nick can be introduced into the endogenous USH2A gene. The double-strand break or single-strand nick can be introduced using one or more rare-cutting endonucleases either in nuclease or nickase formats. The double-strand break or single-strand nicks can be introduced at the site where integration is desired, or a distance upstream or downstream of the site. The distance from the integration site and the double-strand break (or single-strand nick) can be between 0 bp and 10,000 bp.

The methods and compositions described herein can be used to facilitate homology-independent insertion of a transgene into an endogenous USH2A gene. In one embodiment, a transgene can harbor a partial coding sequence of the USH2A gene and flanking rare-cutting endonuclease target sites can be administered to a cell. Following cleavage by the rare-cutting endonuclease, the liberated transgene can be captured during the repair of a double-strand break and integrated within an endogenous USH2A gene. In another embodiment, a linear transgene harboring a partial coding sequence of the USH2A gene can be administered to a cell. The linear transgene can be captured during the repair of a double-strand break and integrated within an endogenous USH2A gene.

The methods described in this document can include the use of ram-cutting endonucleases for stimulating recombination or integrating the donor molecule into the USH2A gene. The rare-cutting endonuclease can include CRISPR, TALENs, or zinc-finger nucleases (ZFNs). The CRISPR system can include CRISPR/Cas9 or CRISPR/Cpf1. The CRISPR system can include variants which display broad PAM capability (Hu et al., Nature 556, 57-63, 2018: Nishimasu et al., Science DOI: 10.1126, 2018) or higher on-target binding or cleavage activity (Kleinstiver et al., Nature 529:490-495, 2016). The gene editing reagent can be in the format of a nuclease (Mali et al., Science 339:823-826, 2013; Christian et al., Genetics 186:757-761, 2010), nickase (Cong et al., Science 339:819-823, 2013: Wu et al., Biochemical and Biophysical Research Communications 1:261-266, 2014), CRISPR-FokI dimers (Tsai et al., Nature Biotechnology 32:569-576, 2014), or paired CRISPR nickases (Ran et al., Cell 154:1380-1389, 2013).

The methods and compositions described in this document can be used in a circumstance where it is desired to modify the coding sequence of USH2A. For example, patients with mutations in exons 1-21 of the USH2A gene (e.g., a guanine deletion, at nucleotide position c.2299) could benefit from the replacement of the 5′ end of the endogenous USH2A coding sequence with a synthetic and WT USH2A coding sequence. In another example, patients with mutations in exons 22-72 of the USH2A gene (e.g., an adenine deletion at nucleotide position c.13140) may benefit from replacement of the 3′ end of the USH2A with a synthetic and WT USH2A coding sequence.

The methods and compositions described in this document can also be used in the production of transgenic organisms or transgenic animals. Transgenic animals can include those developed for disease models, as well as animals with desirable traits. Cells within the animals can be used in combination with the methods and compositions described herein, which includes embryos. The animals can include small mammals (e.g., mice, rats, hamsters, gerbils, guinea pigs, rabbits, etc.), companion animals (e.g., dogs, cats, rabbits, hedgehogs and ferrets), livestock (horses, goats, sheep, swine, llamas, alpacas, and cattle), and primates (capuchin monkeys, chimpanzees, lemurs, macaques, marmosets, tamarins, spider monkeys, squirrel monkeys, and vervet monkeys). The animal can include a zebrafish.

The invention will be further described in the following examples, which do not limit the scope of the invention described in the claims.

### EXAMPLES

### Example 1—Modification of the N-Terminus of the USH2A Protein in Human Cells

The endogenous human USH2A coding sequence (5′ end) was targeted for modification. Three donor molecules were generated to insert a strong constitutive promoter followed by a partial USH2A coding sequence and splice donor sequence. The constructs were designed with arms of homology to facilitate integration by homologous recombination. The first vector, pBA1112-D1, contained a CMV promoter followed by USH2A exons 2-13 and a splice donor sequence. The sequences were flanked by a 483 bp left homology arm and a 900 bp right homology arm. The vector sequence is shown in SEQ ID NO:15 (Table 1) and the corresponding CRISPR nuclease target site is shown in SEQ ID NO:18 (Table 2). To prevent Cas9 from cutting the construct, two synonymous single nucleotide change were included in the PAM sequence. The second vector, pBA1114-D1, contained a CMV promoter followed by USH2A exons 2-15 and a splice donor sequence. The sequences were flanked by a 435 bp left homology arm and a 600 bp right homology arm. The vector sequence is shown in SEQ ID NO:16 and the corresponding CRISPR nuclease target site is shown in SEQ ID NO:19. To prevent Cas9 from cutting the construct, two synonymous single nucleotide changes were included in the target sequence. The third vector, pBA116-D1, contained a CMV promoter followed by USH2A exons 2-20 and a splice donor sequence. The sequences were flanked by a 600 bp left homology arm and a 600 bp right homology arm. The vector sequence is shown in SEQ ID NO:17 and the corresponding CRISPR nuclease target site is shown in SEQ ID NO:20. To prevent Cas9 from cutting the construct, two synonymous single nucleotide changes were included in the target sequence.

TABLE 1
Donor molecules for integration within
the 5′ end of the human USH2A gene

| | | USH2A | Site of | |
|---|---|---|---|---|
| Name | Promoter | exons | integration | SEQ ID NO: |
| pBA1112-D1 | CMV | 2-13 | Following exon 13 | 15 |
| pBA1114-D1 | CMV | 2-15 | Following exon 15 | 16 |
| pBA1116-D1 | CMV | 2-20 | Following exon 20 | 17 |

TABLE 2
CRISPR/Cas9 target sites for targeting double-
trand DNA breaks within the 5′ end of the human
USH2A gene

| | | | SEQ |
|---|---|---|---|
| | | | ID |
| Name | Target | PAM | NO: |
| pBA1113-C1 | GGTCCCAGGTAATGTCCCCA | AGG | 18 |
| pBA1115-C1 | CTGGCCTGTGACCAAGTGAC | AGG | 19 |
| pBA1117-C1 | TAGAAGGACTGAAACCTTAT | AGG | 20 |

CRISPR nucleases, both Cas9 and the gRNA, were generated as RNA and verified for activity in HEK293T cells. CRISPR RNA was delivered to cells by electroporation (Neon electroporation) and gene editing efficiencies were tested by sequence trace decomposition (Brinkman et al., Nucleic Acids Research 42:e168, 2014). Nuclease pBA1113-C1 had approximately 25% activity; nuclease pBA1115-C1 had approximately 40% activity; and nuclease pBA1117-C1 had approximately 20% activity.

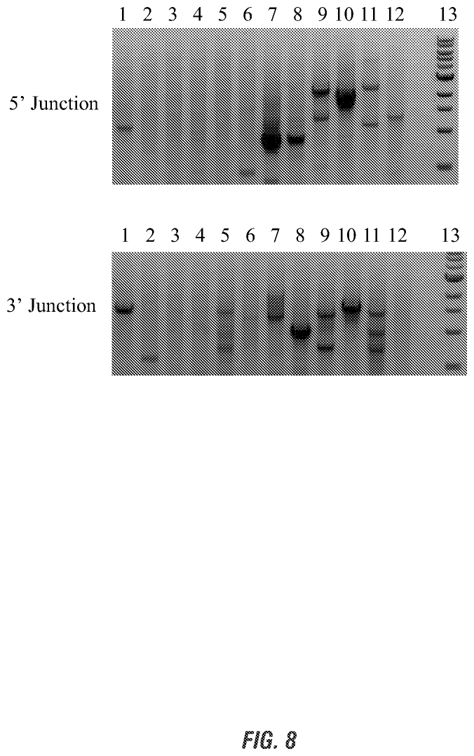

To knockin the USH2A transgenes in the endogenous USH2A gene, both the CRISPR RNA and donor molecules were transfected into HEK293T cells by electroporation. 72 hours post transfection, genomic DNA was isolated. Successful integration of the USH2A transgene was verified by PCR (FIG. 8). Primers were designed to detect the 5′ and 3′ junctions. To detect the 5′ junction of the transgene carried on pBA1112-D1, primers (CCAGCTAATTAATGTATCCATCACC; SEQ ID NO:25) and (AGATGTACTGCCAAGTAGGAAAG; SEQ ID NO:26) were used. To detect the 3′ junction of the transgene carried on pBA1112-D1, primers (GCAAACCCTGTGACTGTGATAC; SEQ ID NO:27) and (GACATAGGGTGGCCATATACC; SEQ ID NO:28) were used. To detect the 5′ junction of the transgene carried on pBA1114-D1, primers (GAATAATGCTGTATTCTCCAACC; SEQ ID NO:29) and (AGATGTACTGCCAAGTAGGAAAG; SEQ ID NO:26) were used. To detect the 3′ junction of the transgene carried on pBA1114-D1, primers (GTTGTGACCAATGCAAAGACC; SEQ ID NO:30) and (CCCAGCAGGCATTCTTAGG; SEQ ID NO:31) were used. To detect the 5′ junction of the transgene carried on pBA1116-D1, primers (GTATTCTACATTCCAATCTCACTGC; SEQ ID NO:32) and (AGATGTACTGCCAAGTAGGAAAG; SEQ ID NO:26) were used. To detect the 3′ junction of the transgene carried on pBA1116-D1, primers (CCACCAGCGGAACTAAATGG: SEQ ID NO:33) and (TGTCTTAACCTCCTTACACATGG; SEQ ID NO:34) were used. The data shows integration of the pBA1112, pBA1114 and pBA1116 transgenes within the endogenous USH2A gene (FIG. 8; Table 3).

TABLE 3
Transfection conditions corresponding to tire
5′ and 3′ junction PCRs

| | | | Expected 5′ | | Expected 3′ | |
|---|---|---|---|---|---|---|
| Lane | Guide | Donor | band | | band | |
| 1 | — | pBA1112-D1 | — | | — | |
| 2 | — | pBA1114-D1 | — | | — | |
| 3 | — | pBA1116-D1 | — | | — | |
| 4 | — | pBA1118-D1 | — | | — | |
| 5 | — | pBA1120-D1 | — | | — | |
| 6 | — | pBA1122-D1 | — | | — | |
| 7 | pBA1113-C1 | pBA1112-D1 | 878 | bp | 2066 | bp |
| 8 | pBA1115-C1 | pBA1114-D1 | 911 | bp | 1004 | bp |
| 9 | pBA1117-C1 | pBA1116-D1 | 1357 | bp | 1436 | bp |
| 10 | pBA1119-C1 | pBA1118-D1 | 2050 | bp | 1679 | bp |
| 11 | pBA1121-C1 | pBA1120-D1 | 2481 | bp | 1041 | bp |
| 12 | pBA1123-C1 | pBA1122-D1 | 1370 | bp | 960 | bp |

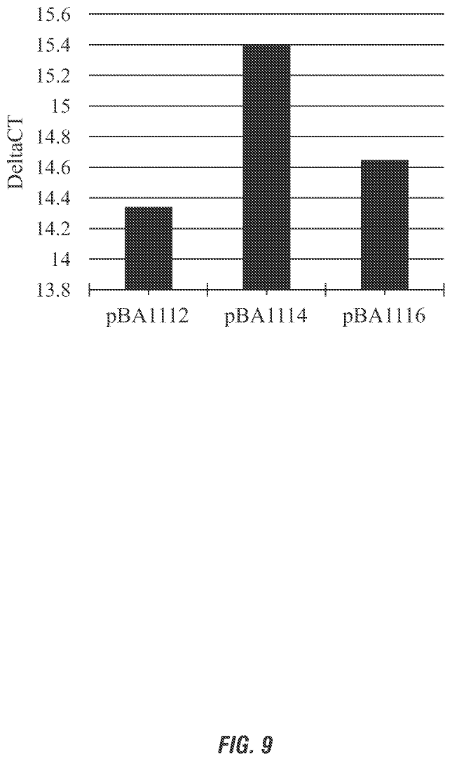

To verify expression of the modified USH2A gene, cDNA was prepared from the population of modified cells. Primers were designed to specifically detect expression from the modified USH2A gene. Primers were designed to bind to the single-nucleotide polymorphisms present within the modified CRISPR target site. To avoid detecting genomic DNA, primers were designed to span an intron. Expression was normalized to an internal control (GAPDH). The results suggest that expression of the modified USH2A gene occurred from targeted integration of pBA1112, pBA1114 and pBA1116 (FIG. 9).

### Example 2—Modification of the C-Terminus of the USH2A Protein in Human Cells

The endogenous human USH2A coding sequence (3′ end) was targeted for modification. Three donor molecules were generated to insert a partial USH2A coding sequence followed by a transcriptional terminator. The constructs were designed with arms of homology to facilitate integration by homologous recombination. The first vector, pBA1118-D1, contained a splice acceptor sequence, USH2A exons 64-72, and a SV40 terminator. The sequences were flanked by a 1500 bp left homology arm and a 1267 bp right homology arm. The vector sequence is shown in SEQ ID NO:49 (Table 4) and the corresponding CRISPR nuclease target site is shown in SEQ ID NO:52 (Table 5). To prevent Cas9 from cutting the construct, a single synonymous nucleotide change was introduced into the PAM site. The second vector, pBA1120-D1, contained a splice acceptor sequence, USH2A exons 63-72, and a SV40 terminator. The sequences were flanked by a 750 bp left homology arm and a 500 bp right homology arm. The vector sequence is shown in SEQ ID NO:50 and the corresponding CRISPR nuclease target site is shown in SEQ ID NO:53. To prevent Cas9 from cutting the construct, a synonymous single nucleotide change was included in the PAM sequence. The third vector, pBA1122-D1, contained a splice acceptor sequence, USH2A exons 61-72, and a SV40 terminator. The sequences were flanked by a 600 bp left homology arm and a 600 bp right homology arm. The vector sequence is shown in SEQ ID NO:51 and the corresponding CRISPR nuclease target site is shown in SEQ ID NO:54. To prevent Cas9 from cutting the construct, two synonymous single nucleotide changes were included in the Cas9 binding sequence.

TABLE 4
Donor molecules for integration within
the 3′ end of the human USH2A gene

| | | USH2A | Site of | |
|---|---|---|---|---|
| Name | Promoter | exons | integration | SEQ ID NO: |
| pBA1118-D1 | CMV | 64-72 | Before exon 64 | 49 |
| pBA1120-D1 | CMV | 63-72 | Before exon 63 | 50 |
| pBA1122-D1 | CMV | 61-72 | Before exon 61 | 51 |

TABLE 5
CRISPR/Cas9 target sites for targeting double-
strand DNA breaks within the 3′ end of the human
USH2A gene

| | | | SEQ |
|---|---|---|---|
| | | | ID |
| Name | Target | PAM | NO: |
| pBA1119-C1 | GCATCAAAGGTGCAATCTCA | GGG | 52 |
| pBA1121-C1 | CACTGAACCCTTGGAGTTAC | AGG | 53 |
| pBA1123-C1 | CATCTTCAGTGACGGGTTCC | TGG | 54 |

CRISPR nucleases, both Cas9 and the gRNA, were generated as RNA and verified for activity in HEK293T cells. CRISPR RNA was delivered to cells by electroporation (Neon electroporation) and gene editing efficiencies were tested by sequence trace decomposition (Brinkman et al., Nucleic Acids Research 42:e168, 2014). Nuclease pBA1119-C1 had approximately 40% activity, nuclease pBA1121-C1 had approximately 20% activity and nuclease pBA123-C1 had approximately 20% activity.

To knockin the USH2A transgenes in the endogenous USH2A gene, both the CRISPR RNA and donor molecules were transfected into HEK293T cells by electroporation. 72 hours post transfection, genomic DNA was isolated. Successful integration of the USH2A transgene was verified by PCR (FIG. 8). Primers were designed to detect the 5′ and 3′ junction. To detect the 5′ junction of the transgene carried on pBA1118-D1, primers (CCTGACTGTACCTCCAACTTC; SEQ ID NO:35) and (AGAATTCACTGCCCAGACCTGAT: SEQ ID NO:36) were used. To detect the 3′ junction of the transgene carried on pBA1118-D1, primers (GCATTCTAGTTGTGGTTTGTCC; SEQ ID NO:44) and (AGTGTfACGTITCCGATGGTG; SEQ ID NO:37) were used. To detect the 5′ junction of the transgene carried on pBA1120-D1, primers (CCATGATAGGGAGTCATCGAAAG: SEQ ID NO:38) and (GTTGCATCAAAGGTGCAATCTC: SEQ ID NO:39) were used. To detect the 3′ junction of the transgene carried on pBA1120-D1, primers (GCATTCTAGTTGTGGTITGTCC; SEQ ID NO:44) and (ATGTGGATTAGCTGCAGAGG; SEQ ID NO:40) were used. To detect the 5′ junction of the transgene carried on pBA1122-D1, primers (GCCAAGCTCAGAGTGAGTITAC; SEQ ID NO:41) and (TCCAGGGTCAGTGTGTAGAG; SEQ ID NO:42) were used. To detect the 3′ junction of the transgene carried on pBA1122-D1, primers (GCATTCTAGTTGTGGTTTGTCC: SEQ ID NO:44) and (ACCAGTAAGCCATAGTGTATGC; SEQ ID NO:43) were used. The data shows integration of the pBA1118, pBA1120 and pBA1122 transgenes within the endogenous USH2A gene (FIG. 8; Table 3).

### Example 3—Modification of the N-Terminus of the USH2A Protein in Human Cells Using CRISPR-Associated Transposases

CRISPR-associated transposase vectors, specifically ShCas12k, are designed to knockin the partial USH2A coding sequences carried on pBA1112, pBA1114 and pBA1116. The CMV promoter is replaced with a splice acceptor operably linked to a viral 2A sequence which is operably linked to the USH2A coding sequences. To design the transgenes for use with ShCas12k (GTN PAM sequence), the homology arms are replaced with the left end (SEQ ID NO:23) and right end sequences (SEQ ID NO:24) of Cas12k transposons. Two vectors are generated: a vector comprising CMV promoters driving expression of tnsB, tnsC and tniQ, and a vector encoding ShCas12k (SEQ ID NO:21). Cas12k guide RNAs are designed to target sequences (GCCTGAGGAAGTCACGAGACCTG; SEQ ID NO:45), (TGCATCAGCAGCCTCCATTGCCC; SEQ ID NO:46) and (CAGCCACTTTGGAAGACAGTTTG; SEQ ID NO:47) for integration of pBA1112, pBA1114 and pBA1116 respectively.

To knockin the USH2A transgenes in the endogenous USH2A gene, the three vectors (ShCas12k, transposon, and tnsB/C/Q vectors) are transfected at equal molar concentrations into HEK293T cells by electroporation. 72 hours post transfection, genomic DNA is isolated and assessed for successful knockin by PCR.

### Example 4—Modification of the N-Terminus of the USH2A Protein (Isoform a and Isoform b) in Human HEK293 Cells

The endogenous USH2A genomic sequence in human HEK293 cells is targeted for modification, specifically exons 1-13, 1-16 and 1-19. Three donor molecules are synthesized along with three CRISPR/Cas9 nucleases. The donor molecules are designed to harbor an hCMV-intron promoter upstream of a synthetic coding sequence for the 5′ end of the USH2A gene. To facilitate targeted integration, donor molecules comprise either homology arms (ranging from 400-600 bp) or cleavage sites for Cas9 (for integration via NHEJ). A list of the donor molecules is shown in Table 6.

TABLE 6
Donor molecules comprising transgenes for integration
within the 5′ end of the USH2A gene

| | | USH2A | Site of | |
|---|---|---|---|---|
| Name | Promoter | exons | integration | SEQ ID NO |
| pBA1005-D1 | hCMV-intron | 2(p)-13 | Exon-intron 13 | 1 |
| | | | junction | |
| pBA1006-D1 | hCMV-intron | 2(p)-16 | Exon-intron 16 | 2 |
| | | | junction | |
| pBA1007-D1 | hCMV-intron | 2(p)-19 | Exon-intron 19 | 3 |
| | | | junction | |

Three CRISPR/Cas9 vectors are designed to introduce double-strand breaks near the predicted site of integration for vectors pBA1005-D1, pBA1006-D I and pBA1007-D1. The gRNA targets are shown in Table 7.

TABLE 7
CRISPR/Cas9 target sites for targeting double-
strand DNA breaks within the 5′ end of the USH2A
gene

| | | | SEQ |
|---|---|---|---|
| | | | ID |
| Name | Target | PAM | NO: |
| pBA1005-C1 | GACATTCCTTTTGTTAACTT | AGG | 4 |
| pBA1006-C1 | TACCCATACAGTGAGTTTAA | GGG | 5 |
| pBA1007-C1 | AACTAATGTCCTTTCAGAAT | TGG | 6 |

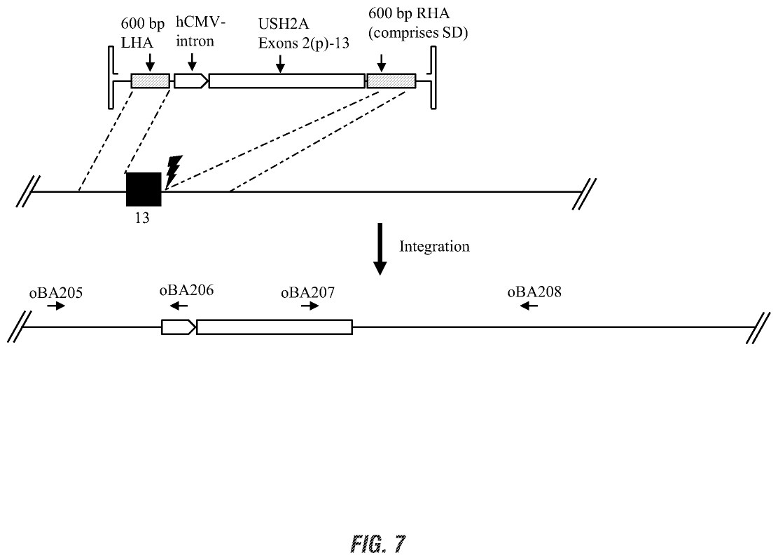

Confirmation of the function of the donor molecules and CRISPR/Cas9 vectors is achieved by transfection of HEK293 cells. HEK293 cells are maintained at 37° C. and 5% CO2 in DMEM high glucose without L-glutamine without sodium pyruvate medium supplemented with 10% fetal bovine serum (FBS) and 1% penicillin-streptomycin (PS) solution 100×. HEK293 cells are transfected with each of the plasmid constructs and combinations thereof using Lipofectamine 3000. Two days post transfection, DNA is extracted and assessed for mutations and targeted insertions within the USH2A gene. Nuclease activity is analyzed using the Cel-I assay or by deep sequencing of amplicons comprising the CRISPR/Cas9 target sequence. Successful integration of the transgene is analyzed using the primers illustrated in FIG. 7.

### Example 5—Modification of the C-Terminus of the USH2A Protein (Isoform b) in Human HEK293 Cells

The endogenous USH2A genomic sequence in human HEK293 cells is targeted for modification, specifically exons 56-72, 58-72, and 60-72. Three donor molecules are synthesized along with three CRISPR/Cas9 nucleases. The donor molecules are designed to harbor a SV40 terminator downstream of a synthetic coding sequence for the 3′ end of the USH2A gene. To facilitate targeted integration, donor molecules comprise either homology arms (ranging from 200-300 bp) or cleavage sites for Cas9 (for integration via NHEJ). A list of the donor molecules is shown in Table 8.

TABLE 8
Donor molecules comprising transgenes for integration
within the 3′ end of the USH2A gene

| | | USH2A | Site of | |
|---|---|---|---|---|
| Name | Terminator | exons | integration | SEQ ID NO: |
| pBA1008-D1 | SV40 | 56-72(p) | Intron 55 | 7 |
| | | | Exon 56 | |
| | | | junction | |
| pBA1009-D1 | SV40 | 58-72(p) | Intron 57 | 8 |
| | | | Exon 58 | |
| | | | junction | |
| pBA1010-D1 | SV40 | 60-72(p) | Intron 59 | 9 |
| | | | Exon 60 | |
| | | | junction | |

Three CRISPR/Cas9 vectors are designed to introduce double-strand breaks near the predicted site of integration for vectors pBA1008-D1, pBA1009-D1 and pBA1010-D1. The gRNA targets are shown in Table 9.

TABLE 9
CRISPR/Cas9 target sites for targeting double-
strand DNA breaks within the 5′ end of the USH2A
gene

| | | | SEQ |
|---|---|---|---|
| | | | ID |
| Name | Target | PAM | NO: |
| pBA1008-C1 | GAGAGTACTCTTAAATGTTT | TGG | 10 |
| pBA1009-C1 | TTGTTCAAGTCTCTTGTGCA | TGG | 11 |
| pBA1010-C1 | AACTACATATTCATACAGAA | GGG | 12 |

Confirmation of the function of the donor molecules and CRISPR/Cas9 vectors is achieved by transfection of HEK293 cells. HEK293 cells are maintained at 37° C. and 5% CO2 in DMEM high glucose without L-glutamine without sodium pyruvate medium supplemented with 10% fetal bovine serum (FBS) and 1% penicillin-streptomycin (PS) solution 100×. HEK293 cells are transfected with each of the plasmid constructs and combinations thereof using Lipofectamine 3000. Two days post transfection. DNA is extracted and assessed for mutations and targeted insertions within the USH2A gene. Nuclease activity is analyzed using the Cel-1 assay or by deep sequencing of amplicons comprising the CRISPR/Cas9 target sequence. Successful integration of the transgene is analyzed using primers within the transgene and within the endogenous USH2A gene (but outside of the extent of any homology arms).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of integrating a transgene into the USH2A gene, the method comprising:
a. administering a rare-cutting endonuclease or transposase targeted to a site within the USH2A gene, and
b. administering a transgene, wherein the transgene comprises at least one component selected from a promoter, 2A sequence, or internal ribosome entry sequence, wherein the at least one component is operably linked to a partial USH2A coding sequence,
wherein the transgene is integrated within the USH2A gene.

2. The method of claim 1, wherein the transposase comprises a Cas12k or Cas6 protein.

3. The method of claim 2, wherein the transposase comprises Cas12k from Scytonema hofmanni or Anabaena cylindrica.

4. The method of claim 1, wherein the rare-cutting endonuclease is selected from a CRISPR nuclease, CRISPR nickase, TAL effector nuclease, TAL effector nickase, zinc-finger nuclease, zinc-finger nickase, or meganuclease.

5. The method of claim 1, wherein the USH2A gene comprises a mutation that causes retinitis pigmentosa.

6. The method of claim 1, wherein the transgene comprises a partial USH2A coding sequence from a functional USH2A gene operably linked to a splice donor.

7. The method of claim 6, wherein the partial coding sequence encodes a peptide produced by exon 2 of a functional USH2A gene.

8. The method of claim 7, wherein the partial coding sequence encodes a peptide as shown in SEQ ID NO:55.

9. The method of claim 7, wherein the transgene is integrated in the USH2A gene within exon 13, within exon 21, or in a region between exon 13 and exon 21.

10. The method of claim 6, wherein the partial coding sequence encodes a peptide produced by exons 2-13 of a functional USH2A gene.

11. The method of claim 10, wherein the partial coding sequence encodes a peptide as shown in SEQ ID NO:13.

12. The method of claim 10, wherein the transgene is integrated within exon 13 or intron 13 of the USH2A gene.

13. The method of claim 6, wherein the partial coding sequence encodes a peptide produced by exons 2-21 of a functional USH2A gene.

14. The method of claim 13, wherein the partial coding sequence encodes a peptide as shown in SEQ ID NO:57.

15. The method of claim 13, wherein the transgene is integrated at the junction of exon 21 and intron 21 of the USH2A gene.

16. The method of claim 1, wherein the transgene comprises at least one of a left and right homology arm, a transposon left end and right end, or one or more rare-cutting endonuclease target sites.

17. The method of claim 1, wherein the transgene is administered to a cell within the retina.

18. The method of claim 1, wherein the transgene is harbored on an adeno-associated virus vector.

19. The method of claim 1, wherein the transgene is administered with lipid nanoparticles.

20. The method of claim 1, wherein the transgene is administered through electroporation.

21. The method of claim 1, wherein the promoter is a tissue specific promoter, inducible promoter, an USH2A promoter, or constitutive promoter.

22. A method of integrating a transgene into the USH2A gene, the method comprising:
a. administering a rare-cutting endonuclease or transposase targeted to a site within the USH2A gene, and
b. administering a transgene, wherein the transgene comprises a partial USH2A coding sequence operably linked to a terminator.
wherein the transgene is integrated within the USH2A gene.
